(19) United States
(12) Patent Application Publication
(10) Pub. No.: US 2005/0170510 A1
(43) Pub. Date: Aug. 4, 2005

(54) Device and method for controlled electroporation and molecular delivery into cells and tissue

(76) Inventors: Yong Huang, Milpitas, CA (US); James W. Borninski, Berkeley, CA (US); Laura T. Mazzola, Redwood City, CA (US)

Correspondence Address: Laura Mazzola;Excellin Life Sciences, Suite 2050, 1455 Adams Dr, Menlo Park, CA, 94025, US

(21) Appl. No.: 11/007,661

(22) Filed: Dec. 7, 2004

Related U.S. Application Data
(60) Provisional application No. 60/528,147, filed on Dec. 8, 2003.

(51) Int. Cl. C12N 13/00; C12N 5/08

(52) U.S. Cl. 435/459; 435/368

(57) ABSTRACT

In biology and biotechnology, electroporation is an important technique for introducing entities (DNA, RNAi, peptides, proteins, antibodies, genes, small molecules, nanoparticles, etc.) into cells. Applications range widely from genetic engineering to regenerative medicine to drug delivery. It has been demonstrated that the electrical currents flowing through cells can be used to monitor and control the process of electroporation for biological and artificial cells. In this application, a device and system are disclosed which allow precise monitoring and controlling electroporation of cells and cell layers, with examples shown using adherent cells grown on porous membranes.

### CROSS-REFERENCES TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF INVENTION

(1) Field of the Invention

This invention is related to the field of cell electroporation and molecular delivery in general, which specific reference to controlling electroporation in biological and synthetic cells, tissue, and lipid vesicles.

(2) Description of the Related Art

Unlike the present invention, most electroporation devices electroporate cells while they are in solution (suspension). One problem incumbent with electroporation of cells in suspension is that it is not possible to measure, much less control, the voltage drop over any individual cell. Moreover, due to inhomogenieties in the suspension and on the electrodes, individual cells will see a broad range of voltages—in essence the biological cell population experiences significant inhomogeneities in the localized electric field, resulting in significant differences in observed electroporation from cell to cell. Thus, cell death by irreversible electroporation is common, that is, electroporation during which the voltage is sufficiently high to irreversibly damage the cell membrane. In the case of traditional electroporation (FIG. 18: EP1-EP4 indicate typical results), typically less than 50% of the cells survive the process and the transport efficiency is less than 50% for the remaining cells. At the other extreme, a fraction of the overall cell population experiences no effective electroporation due to insufficient magnitude of their local electric field. For this case, traditional electroporation produces a highly inhomogeneous result with less than 25% overall efficiency.

Of significant benefit would be the enablement of a controlled process to large cell populations (hundreds to tens of thousands to millions of cells). Many cell-based assay techniques currently used in the biopharmaceutical industry require large cell populations for drug discovery and screening, which depend upon a homogeneous and uniform cell population. Unfortunately, there exists no technique for processing large populations of cells that ensures highly efficient and uniform electroporation while maintaining high cell viability. Recent developments in the area of controlled electroporation have shown that immobilization of a cell to a solid, porous support can improve the efficiency of electroporation while maintaining cell viability, as demonstrated in applications of electroporation of a single cell. However, this technique is deficient in that a homogeneous electroporation process cannot be achieved in large cell populations. Homogenous, simultaneous electroporation of large cell populations requires control of each cell's local electric field, or at least by ensuring a uniform and homogeneous local electric field over the entire cell population. For large cell populations to be processed simultaneously, a high degree of uniformity and homogeneity of the local applied field must be achieved. The problems and deficiencies of current methods in electroporation of cell populations, in particular with large cell populations, are addressed by the present invention.

### BRIEF SUMMARY OF THE INVENTION

In the present invention, electrical current is directed to flow through biological cells, making it possible to accurately measure, and thus precisely control, the voltage over the cells—ensuring a uniform, homogeneous field applied to the cell or cell population. Since the present invention allows more precise control of the voltage applied to cells, it provides a means of ensuring that cells are not killed during electroporation.

Furthermore, the voltages applied to the electroporation electrodes (3 and 15) in the present invention can be more than two orders of magnitude lower than those used in electroporation systems where the cells are in suspension. Since the cells present a large electrical impedance, the bulk impedance of the electrolyte becomes negligible, and most of the voltage applied to the electroporation electrodes (3 and 15) drops over the cells. This ‘focusing’ of the electric field permits application of electroporation voltages to the electroporation electrodes (3 and 15) that is very close to the actual cross-cell voltage required to initiate electroporation.(roughly between 0.3V and 1.0V). Lower voltages in turn reduce the complexity, size and cost of the power amplifier (21), and also allow electroporation pulses of arbitrary shape and duration without adding complexity to the power amplifier (21).

The described process and apparatus for controlled electroporation provides a universal method for intracellular molecular delivery simultaneously for small and large cell populations. This combination of highly efficient molecular transport with high cell viability is unique, particularly with adherent cells and sensitive, primary (patient-derived) cells. This effect is captured in FIG. 18, where the methods of traditional electroporation and lipofection are compared to the results achieved using the herein described device for controlled electroporation. The axes represent the relative rates of cell survival (viability) and transport efficiency (delivery). The diameter of each “bubble” represents the overall efficiency for primary cell transfection (here using MDCK cell data), a factor of the viability and delivery for each technique. (The larger the bubble diameter, the greater the overall efficiency.)

The traditional methods of mechanical cell delivery (electroporation, ballistics and microinjection) generally cause significant cell death due to irreversible membrane rupture. In the case of traditional electroporation (FIG. 18: EP1-EP4 indicate typical results), generally less than 50% of the cells survive the process and the transport efficiency is less than 50% for the remaining cells. For this case, traditional electroporation often has less than 25% overall efficiency. Chemical methods of cell delivery such as lipofection (FIG. 18: LF1-LF4 indicate typical results) show little immediate harm to cell viability, but these methods often have very low transport efficiency with tissue-derived cells and other primary cells, typically less than 30% overall efficiency. The controlled electroporation process typically produces greater than 80% overall efficiency, and in many cases greater than 90% overall efficiency—produced by the >90% cell viability and >80% transport efficiency of this process. This very high overall efficiency provides a new utility for cell engineering, particularly important for delicate cells and primary cells, in that the engineered cells can be studied without tedious and time consuming cell sorting to remove dead or unprocessed cells. Particularly because of the high cell viability of the controlled electroporation process, this method and device enables new research in areas where cells are precious or rare, or where speed and efficiency in processing is critical.

The controlled electroporation process and apparatus provides great benefit in the ability to observe transient transfection in hard-to-transfect cells, as the researcher can now observe gene expression within a few hours the transfection event. Without this technique, researchers may spend more than a month developing each stably-transfected cell type to ensure a consistent signal for cell detection; the other possibility is to spend days sorting the cells to isolate the transfected cells, racing against time to take data before the expression levels fade.

Primary cells greatly benefit from the process of controlled electroporation. For example, in the area of regenerative medicine, this process and device can be used to import genes, proteins and other material to induce differentiation or selective regeneration of stem cells, nerve cells and other critical primary cells of interest. These cells are useful both for research and development, but also may be used as a source for tissue generation for re-implantation and regeneration. As another example, blood products may be infused with drugs, proteins and other therapeutic compounds—then infused back into the body as cell-based drug carriers. In the case of blood products (including, but not limited to platelets, white blood cells and red blood cells), these cells often aggregate at the sites of trauma, tumors or blood clots. In this way, one may infuse the blood cells with therapeutics for targeted drug delivery, using the cells as natural targeting agents. Using the described process of controlled electroporation, the high level of efficiency in molecular delivery allows a robust and reliable process for targeted cell therapeutics, without significant loss in cells and without the need to sort or screen cells before use.

### BRIEF DESCRIPTION OF THE SEVERAL VIEWS OF THE DRAWINGS

FIG. 1a is a schematic illustration of the electroporation device design and electronics configuration. FIG. 1b is the system diagram of the feed-back control electronics. FIGS. 1c-e depict various configurations of loop-gain adjustment circuits

FIG. 2 illustrates methods for blocking micro pores on a porous membrane by a) forming a confluent cell layer by growing adherent cells on the porous membrane and b) forcing suspended cells/lipid vesicles to block the pores with pressure field.

FIG. 3 shows a method for blocking pores that are not covered by cells with non-conductive particles using pressure a) before suction pressure is applied and b) when suction pressure is applied.

FIG. 4 depicts waveforms of various electroporation pulses: a) single step pulse, b) three-step pulse, c) four-step pulse, d) sinousoid pulse, e) sinusoid-superpositioned step pulse.

FIG. 5 is a schematic of controlling electroporation in tissue slice with a four-electrode device.

FIG. 6 illustrates a typical three-step electroporation pulse used to measure electrical resistance of cells before, during and after electroporation.

FIG. 7 contains the electrical responses of fibroblast cells grown on a porous membrane under three-step electroporation pulses: a) the first pulse and b) second pulse applied 1 minute later.

FIG. 8 shows the electrical responses of MDCK cells grown on a porous membrane under a four-step electroporation pulse: a) the first pulse and b) second pulse applied 1 minute later.

FIG. 9 illustrates the electrical responses of mouse liver tissue slice under three-step electroporation pulses: a) fresh liver tissue, 2.0 mm thick sample, b) fresh liver tissue, 2.5 mm thick sample and c) dead liver tissue, 2.5 mm thick.

FIG. 10 is a fluorescent image of electroporated MDCK cells stained with PI (Transfection efficiency >90%).

FIG. 11 is a fluorescent image of MDCK cells stained with PI dye after electroporation, showing virtually no cell death induced by the controlled electroporation

FIG. 12 is a fluorescent image of electroporated differentiated MDCK monolayer expressing GFP reporter gene (expression efficiency >95%).

FIG. 13 is a fluorescent image of electroporated satellite stem cells expressing GFP reporter gene (expression efficiency >95%).

FIG. 14 is a fluorescent image of electroporated fibroblast cells expressing GFP reporter gene (expression efficiency >90%).

FIG. 15 contains images of a) fibroblast cells before transfection and b) Myotube cells by transfection of fibroblast cells with MyoD gene (˜40 Kb).

FIG. 16 is a fluorescent image of MDCK epithelial cells after transfection with fluorescenated siRNA (FITC-siRNA) (Transfection efficiency >95%).

FIG. 17 is a fluorescent image of MDCK epithelial cells after transfection with fluorescenated anti-mouse antibody (Transfection efficiency 98.4%).

FIG. 18 is a bubble chart comparison of controlled electroporation performance (upper right corner) vs. traditional electroporation (horizontal stripes) and lipofection (vertical stripes). Bubble diameter indicated the overall efficiency of the process (delivery×viability).

### DETAILED DESCRIPTION OF THE INVENTION

### Device Configuration

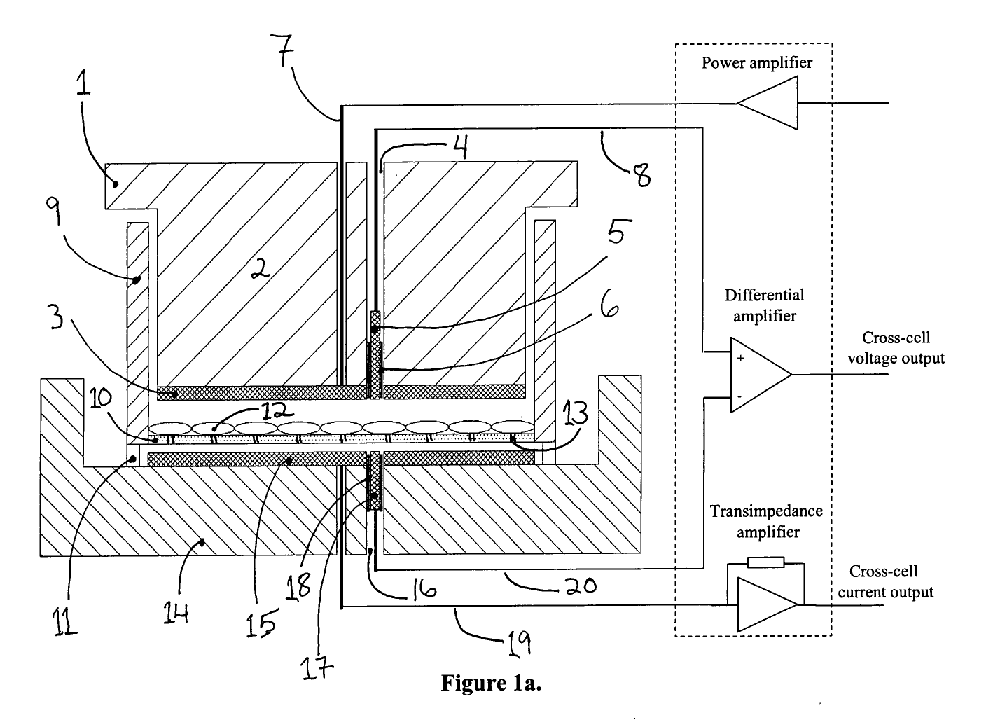

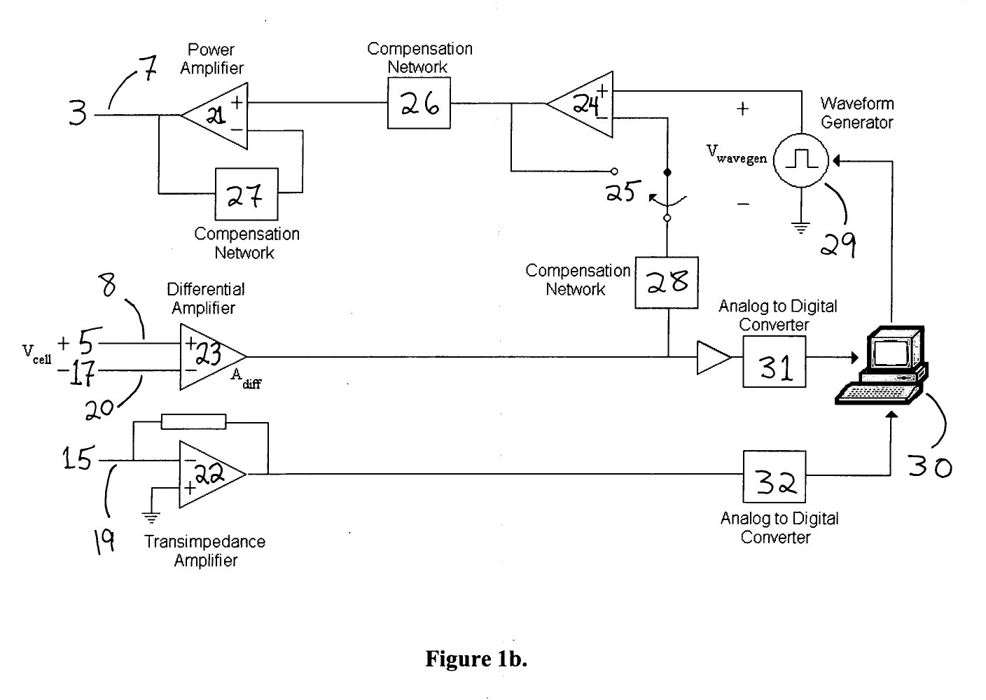

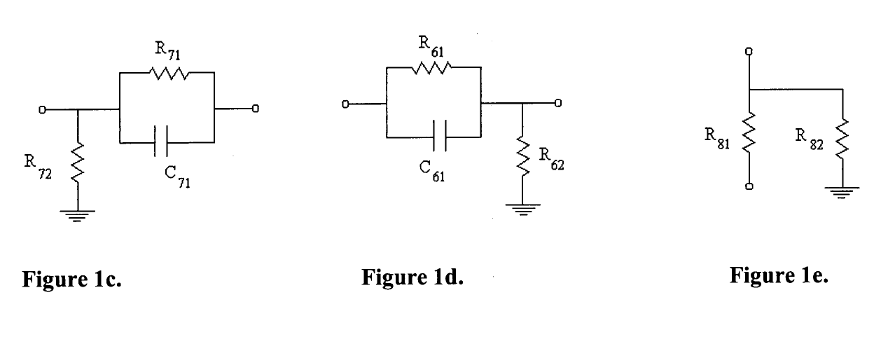

FIG. 1a shows the cross-section schematic of a device as well as the electronic configuration for monitoring and controlling electroporation of cells (including but not limited to biological cells, lipid vesicles, cell cultures, cell monolayers, spheroids, biological tissue and tissue slices and any combination thereof) on porous membranes. The device consists of three parts: The top unit (1), the middle cup (9) and the bottom chamber (14). The middle cylindrical cup has a thin, non-electrically conductive and porous membrane (10). The cup rests on feet (11) to keep the membrane (10) from touching the bottom chamber (14). Alternately, a flange along the top rim of the cup (9) allows the cup to hang from a ledge built into the bottom chamber, such that the membrane (10) is separated at a desired distance from the bottom chamber (14). A top electroporation electrode (3), typically made of silver and silver chloride, is attached to the base of the top unit body (2) as shown in the figure. The surface of the top electroporation electrode (3) has roughly the same area and shape as the surface of the porous membrane (10). In practice, and as shown in FIG. 1a, it may be necessary to make it slightly smaller than the membrane due to constraints imposed by cup (9). In an ideal embodiment, the top electroporation electrode (3) would actually be larger than the membrane. A small hole (4) is provided in the top electroporation electrode through which a probe electrode (5) is inserted. Nonconductive filling (6) is used as a spacer for the probe electrode to prevent electrical connection between the two electrodes. Electrical wires (7, 8) connect the top electroporation electrode (3) and the probe electrode (5) to external electronic apparatus.

The bottom chamber consists of a body (14) and a bottom electroporation electrode (15) attached to the inside of the chamber. While it may be possible to use an identical design for both the top and bottom electroporation electrodes, in practice, the mechanical dimensions are likely to differ. For example, as shown in FIG. 1a, it may be desirable to make the surface area of the bottom electroporation electrode (15) larger than that of the top electroporation electrode (3) in order to reduce fringing effects when a voltage is applied across these electrodes. As in the top unit, a probe electrode (17) is inserted in the bottom electroporation electrode (15) through a hole (16) and nonconductive filling (18) is used to insulate the electrodes. Electrical wires (19 and 20) provide electrical access to the two electrodes.

In an experiment, biological entities block substantially all of the micro pores (13) on the porous membrane (10), in some cases forming a continuous layer of cells across the membrane. The middle cup is placed in the bottom chamber (14). The proper amount of conductive electroporation buffer is injected in both the bottom chamber and the cell cup. Entities for molecular transfer can be placed in either the upper or lower electroporation reservoir, depending on the desired polarity of the applied electric field and the polarity of the cell and entities. (The two reservoirs may also contain different entities and/or a plurality of entities at different concentrations.) The top unit is inserted in the cell cup. By design, the electroporation electrodes on bottom chamber (15) and the top unit (3) maintain a fixed distance to the cell and the porous membrane and the intervening space is filled with a conductive electroporation buffer. The top electroporation electrode (3) is connected to the output of a power amplifier (21) via the wire (7). The bottom electroporation electrode is connected to a transimpedance amplifier (22). The top measurement electrode (5) and bottom measurement electrode (17) are connected to the two inputs of a high input-impedance differential voltage amplifier (23) through electrical wires (8) and (20) respectively.

During an electroporation experiment, electrical pulses are applied to the cells through the two electroporation electrodes (3 and 15). When the cells are electroporated, electrical current flows through the cell membrane(s) through field-induced pore formation. During this process, entities can be delivered into the cell by transport mechanisms including passive transport (diffusion), electrophoretic force, electrokinetic or electroosmotic flow, or any combination thereof. The magnitude of this electrical current is dependent on the degree of electroporation of the cells. This electroporation-induced electrical current can be measured with the transimpedance amplifier (22) and can be used to monitor the process of cell electroporation. In addition to the current measurement, the two measurement electrodes are used to precisely measure the voltage drop across the cell layer during the electroporation process. Because of the voltage drops at the electrode-electrolyte interfaces, the voltage applied to the two electroporation electrodes (3 and 15) is not the same as the voltage across the cell or vesicle layer. The two measurement electrodes (5 and 17) are connected to the high input-impedance amplifier (23). Thus, since no current flows through these electrodes (5 and 17), there is no voltage drop over the electrode-electrolyte interfaces, and the differential voltage between them provides accurate readings on the voltage across the cell layer. Precise electrical impedance of the cell layer can thus be calculated, for example, by a computer, with cross-cell voltage measurement and cross-current measurement; the impedance measurement precisely reveals the degree of electroporation of the cell or cell layer since cell membrane impedance is a function of the extent of membrane electroporation. The electrical membrane impedance can be used as feedback to fine-tune electroporation pulses, in order to achieve highly controlled electroporation of the cells as well as for monitoring the recovery process of the cell membranes after electroporation.

Methods for Blocking Micro Pores on a Porous Membrane with Cells

Effective blocking of the micro pores on the porous membrane (for example, with cells or other pore-blocking matter) is critical to achieve highly controlled electroporation using the device described above. Pores that are not blocked by cells produce parasitic currents pathways when electroporation pulses are applied, which reduces accuracy in trans-membrane current measurement and also deteriorates the ‘focusing’ effect of this configuration, resulting less effective electroporation of the cells. Moreover, unblocked pores which are distributed non-uniformly across the membrane can result in electric field asymmetries across the membrane surface. (It is important to note that while incomplete coverage of pores may result in these adverse effects, the described apparatus can still achieve electroporation at applied voltages much smaller than those used for traditional electroporation.)

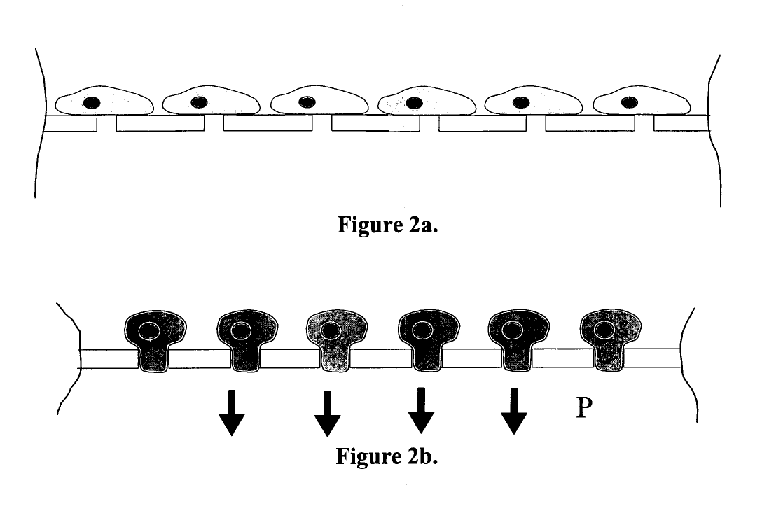

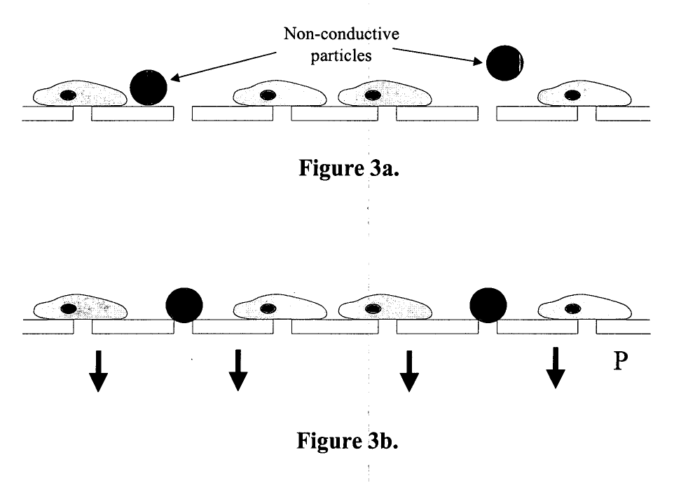

There are several ways to effectively block micro pores such that electrical currents are forced to flow through cells during electroporation process. FIG. 2.a illustrates the first method, which is the formation of a confluent layer of adherent cells to cover a porous membrane (10). In this process, adherent cells are cultured on a porous membrane which is constructed of materials such as polycarbonate, PET or PTFE, and is tissue culture treated and/or coated with cell growth permissive coatings (including but not limited collagen, fibronectin, or polylysine or any combination thereof). When cells grow into an interconnected monolayer that covers the entire porous membrane, they effectively electrically block all micro pores. In this scenario, there is insignificant current flowing between the two electroporation electrodes (3 and 15) if the applied electrical voltage is not large enough to induce electroporation in the cells; since the cell membranes have very high electrical impedance, current cannot flow through them to the micro pores, which in turn provide the only current pathways between the two electrodes. In practice, a small leakage current will develop due to imperfect sealing of the cells to the pores, as well as uncovered pores. In many cases, it is difficult or not desirable to grow cells into a 100% confluent layer. This may result in many uncovered micro pores, which as discussed previously can be detrimental to device performance. To solve this problem, non-conductive substances, such as micro glass beads, can be added to block the uncovered pores through various mechanisms. As illustrated in FIG. 3, one method to achieve this is through generating a pressure difference between the two sides of the porous membrane to pull the substances toward the uncovered pores and block/clog them.

Suspension cells normally do not attach to substrates and form an adherent layer. For these kinds of cells, a mechanical means is required for sealing of micro pores. One such mechanical means is generating a pressure difference between two sides of the porous membrane such that the suspended cells are pulled toward pores; thus the deformed cells can effectively block the micro pores as illustrated in FIG. 2.b. Because excessive pressure difference can cause mechanical damage to cell membrane, the pressure must be properly regulated so as to produce a good seal between cells and pores, but avoid damaging the cells. This pressure difference may be generated by: providing a seal between the top unit (1) and the cup (9), and applying a positive pressure through a small hole in the top unit; providing a seal between the bottom chamber (14) and the cup (9), and a negative pressure is applied through a small hole in the bottom chamber; or some external device prior to insertion of the cup (9). In the former two cases, the pressure may be applied throughout an experimental procedure. Another mechanical means of moving the cells into a position whereby they block the micro pores is centrifugation; the entire cup (9) along with cells or lipid vesicles in a suspension of liquid is placed in a centrifuge such that the cells are forced through the liquid to coat the membrane under the action of the centrifuge. These mechanical techniques, pressure differential or centrifugation can be used together. They can also be used in conjunction with an adherent cell layer resulting from cell growth, as described above; for example, it may be necessary to block the pores in areas not covered by the adherent cell layer. Either of these mechanical techniques, pressure differential or centrifugation, can be combined with the use of non-conductive substances, such as micro glass beads, to block any uncovered pores, as described above. For example, in the case where it is desired to electroporate cells which are fewer in number than pores on the porous membrane (10), an effective experimental protocol may consist of adding the cells in liquid suspension to the cup (9); applying a pressure differential to move the cells to block pores; adding sufficient micro or nano-sized inert particles to cover the remaining pores; and applying a pressure differential to move the particles to plug the remaining pores.

The percentage of pores that are effectively blocked can be evaluated by simply measuring the overall impedance of the cell-covered porous membrane. This is because when a micro pore is blocked by a cell whose membrane impedance is very large, the effective impedance of this cell-pore unit is far larger than that of an uncovered micro pore. Therefore, the more pores that are effectively covered by cells or blocked by non-conductive substances, the larger the overall impedance of the cell-membrane complex will be. The correlation between pore coverage and overall impedance can be readily established; by applying a low voltage to electroporation electrodes (3 and 15) which does not induce electroporation in cells, and by measuring the corresponding impedance, the effectiveness of pore-coverage can be evaluated. This impedance measurement can be very helpful in later determination of the optimal electroporation voltages.

### Intrinsic Cell Membrane Potential

Due to charge accumulation within biological cells and on cell membrane surfaces, a cell membrane can be described as having a built-in potential. During an electroporation experiment, this potential contributes to, or subtracts from, the externally supplied voltage; thus, highly controlled electroporation requires knowledge of the membrane built-in potential. The present invention allows measurement of this intrinsic cellular potential prior to electroporation. For such a measurement, the top and bottom electroporation electrodes (3 and 15) must be electrically disconnected from the power amplifier (21) and the transimpedance amplifier (22) respectively. These electrodes (3 and 15) may be allowed to float. Alternatively, the top electroporation electrode (3) may be connected to the top measurement electrode (5) and the bottom electroporation electrode (15) may be connected to the bottom measurement electrode (17). In the case that the differential amplifier (23) common mode rejection is inadequate, it may be necessary to connect either the top (5) or the bottom (17) measurement electrode to a defined potential, such as the reference ground of the differential amplifier (23).

### Transepithelial/Transendothelial Impedance Measurement

Studies of the barrier and transport functions of epithelia and endothelia commonly rely on measurements of the electrical impedance of monolayers of such cells. This property is termed the transepithelial or transendothelial impedance. As described above, the present invention is capable of performing such impedance measurements. Thus, the present invention is uniquely qualified to assess barrier and transport function changes as a result of electroporation, as well as barrier and transport function changes due to the transfer of any foreign substance into or through the cells during electroporation.

Furthermore, there is a unique aspect of cell orientation that can be exploited by using the above described method and device for controlled electroporation. The porous membrane provides a natural support for tissue-derived cell growth, and thus allows a more natural state of the cell or cell layer for in situ electroporation. The device also provides a means for controlling orientation of the cell and/or cell layer. By way of example, cells like the MDCK epithelial cells are known to differentiate as a function of development and cell density—they naturally develop orientation (cell polarity) with apical, basal lateral membrane polarization, which differ in lipid and protein composition. We have observed a vector-dependence to the electroporation performance with MDCK cells, meaning that direction of the applied electric field may depend on the differentiated membrane orientation, i.e. apical-to-basal lateral vs. basal lateral-to-apical applied fields. Given that most tissues and tissue-derived cells have defined growth vectors (motility) and orientation preferences, the device has novel use in determining and optimizing cell engineering for adherent cells and tissue.

### Methods for Using Feedback for Controlled Electroporation

As mentioned above, the voltage applied by the power amplifier (21) to the electroporation electrodes (3 and 15) is not the same as that seen by the cells. However, the voltage measured by the differential amplifier (23) through the measurement electrodes (5 and 17) is an accurate representation of the voltage that drops over the cells. Thus, the operator of the present invention can use the voltage produced by the differential amplifier (23) as guidance, or ‘feedback,’ when attempting to apply a desired voltage to the cells; specifically, the operator may increase the voltage applied by the power amplifier (21) until the voltage measured by the differential amplifier (23) reaches the desired value. Alternately, the operator can use the current measured by the transimpedance amplifier (22) as feedback; as described above, the magnitude of the electrical current is dependent on the degree of electroporation of the cells. Thus, the operator may increase the voltage applied by the power amplifier (21) until the current measured by the transimpedance amplifier (22) reaches the desired value. Finally, the operator can use the impedance measurement of the cells as feedback. As described above, precise electrical impedance of the cell layer is calculated with cross-cell voltage measurement from differential amplifier (23) and cross-current measurement from transimpedance amplifier (22). The impedance measurement precisely reveals the degree of electroporation of the cell layer since cell membrane impedance is directly dependent on the extent of membrane electroporation Thus, the operator may increase the voltage applied by the power amplifier (21) until the calculated cell layer impedance decreases to the desired value.

The above paragraph describes the manual use of measured data by the operator of the present invention as feedback for achieving desired results. Specifically, since the voltage applied to the cells is not the same as that applied to the electroporation electrodes (3 and 15), the operator is required to adjust the voltage applied to the electroporation electrodes (3 and 16) until the voltage applied to the cells, as measured by the differential amplifier (23) reaches the desired value. In this case, the electronic circuit is configured in an open-loop fashion, as shown in FIG. 1a. An alternate technique is to design the electronic circuitry in a closed-loop configuration in order to force the voltage between the measurement electrodes (5 and 17) to a desired value. FIG. 1b depicts one such configuration. Negative feedback is accomplished through the use of an operational amplifier (24). A switch (25) allows the circuit to be configured as closed-loop or open-loop. The position of the switch (25) shown in FIG. 1b is the position required for closed-loop operation. The closed-loop circuit may become unstable due to poles contributed by: amplifiers 21, 23 and 24; the electrodes 3, 5 and 17; and the cells or cell layer. Three optional compensation elements (26, 27 and 28) can be used to ensure the stability of the closed-loop circuit. 26 and 27 may be configured as shown in FIGS. 1c and 1d respectively, in which case they would both serve as phase lead elements, in addition to allowing adjustment of loop gain. An example configuration of 28, shown in FIG. 1e, is used for adjusting loop gain. In the example configuration presented above, the voltage across the cells is directly controlled by the waveform generator (29) output according to the following relationship:

$${V}_{cell} \approx {V}_{wavegen} \times \frac{1}{{A}_{diff}} \times \frac{{R}_{81} + {R}_{82}}{{R}_{82}}$$

Where A_{diff} is the gain of the differential amplifier (23), V_{wavegen} is the output of the waveform generator (29) and V_{cell} is the voltage across the cell layer.

In one realization of the invention, the waveform generator (29) is controlled by a computer (30). The output of the differential amplifier (23), which represents the voltage across the cells, is converted to digital by the analog to digital converter 31, while the output of the transimpedance amplifier (22), which represents the current flowing through the cells, is converted to digital by the analog to digital converter 32. 31 and 32 in turn pass on the digital information to the computer (30). As described above, the electrical impedance can thus be calculated using a computer. This impedance measurement can in turn be used by computer software to change the output of the waveform generator (29). Thus, the voltage applied to the cells can be adjusted to achieve a desired cell impedance; for example, if the calculated impedance is higher than the desired impedance, the computer (30) can increase the magnitude of the output of the waveform generator (29), thus increasing the voltage applied to the cells. The computer will continue to increase the voltage applied to the cells until the degree of electroporation of the cells results in the impedance decreasing to the desired value.

### Description of Electrical Pulses

As described above, the unique configuration of the present invention allows electroporation voltage pulses more than two orders of magnitude smaller than those used for electroporation of cells in suspension; this in turn allows generation of arbitrary pulse shape and duration without adding complexity to the power amplifier (21). Note that the polarity of a pulse is defined as follows: a positive pulse is one in which the potential of the top electroporation electrode (3) is positive with respect to the potential of the bottom electroporation electrode (15).

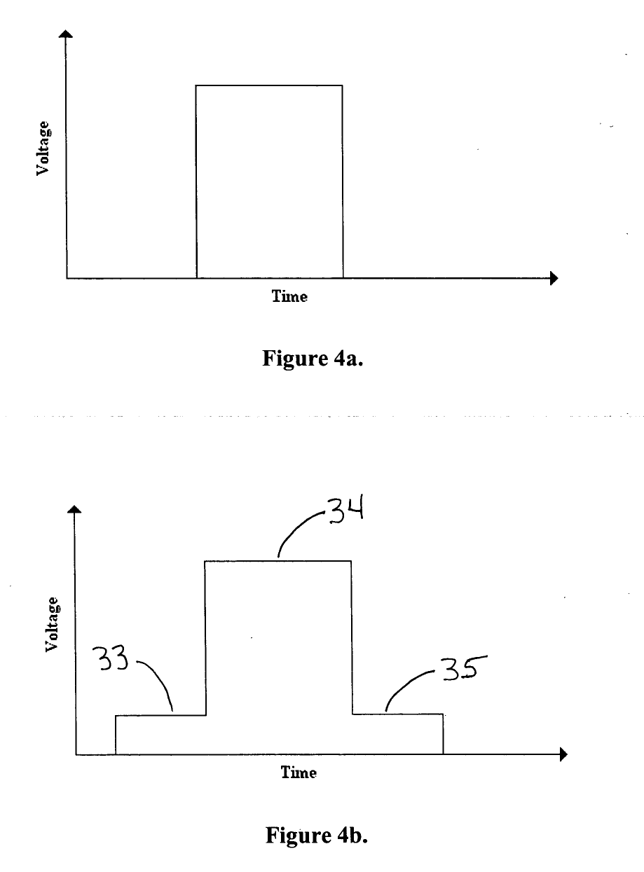

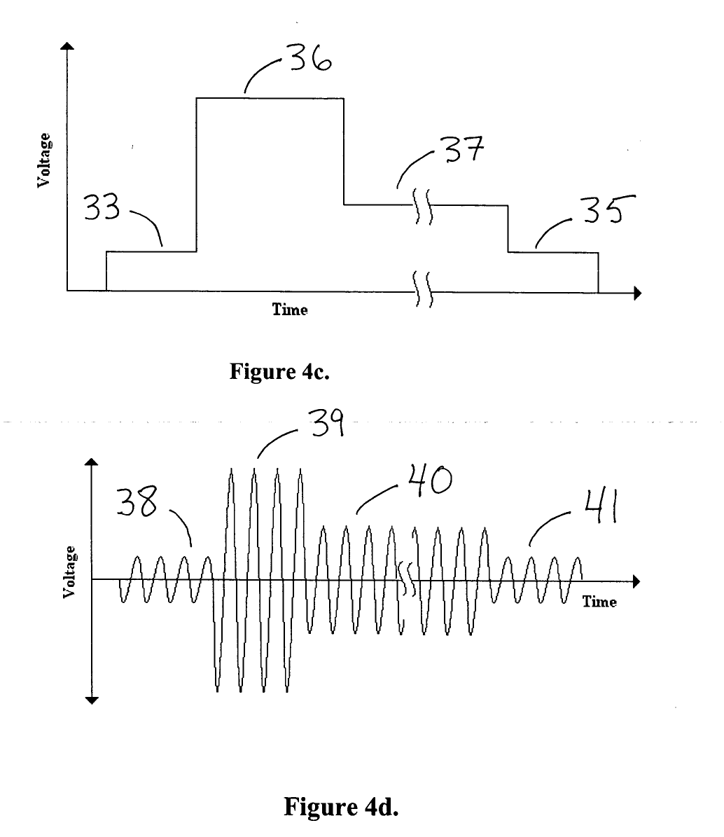

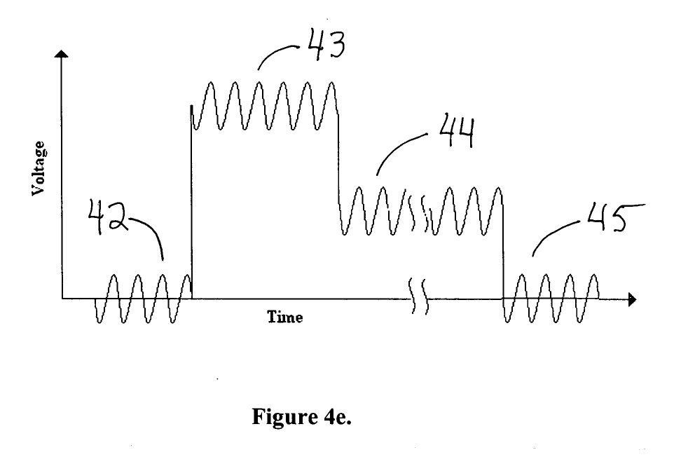

The simplest such pulse is a step pulse, that is, a step from ground potential to some constant voltage, which is maintained for some period of time, followed by a step from this constant potential back down to ground potential. Such a pulse is shown in FIG. 4.a. In order to initiate reversible electroporation, the potential drop across the cell layer, as measured by the differential amplifier (23) through the measurement electrodes (5 and 17), should be roughly greater than about 200 mV and less than about 1000 mV, depending on cell type and charge. (Note that due to intrinsic cell charge, the absolute value of the threshold may be different for opposite polarity pulses.) To achieve this, the voltage that must be generated by the power amplifier (21) is typically less than 20V. The width of this pulse should be greater than approximately 100 ms and less than approximately 3000 ms, as longer pulses may cause irreversible electroporation. It may be desirable to immediately precede and follow this step pulse by contiguous step pulses of lower amplitude, as shown in FIG. 4.b, where this amplitude is sufficiently low (20-50 mV) such that it does not cause electroporation of the cells. The low amplitude pulse (33) preceding the electroporation pulse (34) allows measurement of the impedance of non-electroporated cells. This measurement serves as comparison for the impedance measured during the electroporation pulse (34); a decrease in impedance during the electroporation pulse (34) as compared to that measured during the pre-electroporation pulse (33) indicates that electroporation has taken place. The low amplitude pulse following electroporation (35) allows assessment of the recovery of cells from electroporation; an increase in impedance during the post-electroporation pulse (35) as compared to that measured during the electroporation pulse (34) indicates that the cells have begun to recover from electroporation.

As described above, the electroporation pulse (34) should be limited to ensure cell viability and to protect the electrodes. However, it may be desirable to extend the time in which mass transfer can take place, and to help drive mass transfer through electrophoresis. This would appear to be particularly important given the direct current (DC) nature of the pulses described. It appears to us that, once cells are electroporated, the potential required to maintain a given degree of electroporation is in the range of 100 mV to 500 mV, and as such is much lower than the threshold value for initiation of electroporation. When set in this potential range, a pulse may be several seconds long. Therefore, it may be advantageous to divide the electroporation pulse (34) from FIG. 4.b into two segments, as shown in FIG. 4.c. The first part of the electroporation pulse (36), is intended to initiate electroporation. The second part of the electroporation pulse (37) has a lower amplitude than the first part (36), and is intended to maintain electroporation. Note that the two portions of the electroporation pulse need not be the same polarity. For example, if the intrinsic charge of the cell membrane is positive, it may be desirable to make the first part of the electroporation pulse (36) negative. However, if the molecule to be transferred is, for example, positively charged, it may be advantageous to make the second portion of the electroporation pulse (37) positive in order to assist in electrophoresis.

Under certain circumstances, a sinusoidal pulse, defined as a finite number of periods of a sinusoid with a constant amplitude and frequency, is preferred over the step pulses described above. For example, a sinusoidal pulse prevents deterioration of the electroporation electrodes (3 and 15). Moreover, the step pulses described above may result in polarization of the electrodes, which in turn could lead to measurement errors. Finally, a sinusoidal pulse may result in more efficient transfer of molecules or in increased cell viability for certain cell types. The cell or lipid vesicle layer can be modeled as a resistor in parallel with a capacitance, and thus the impedance of the layer will have a low pass filter response. During electroporation, the resistance of the cell layer will decrease while the capacitance will remain largely unchanged. Thus, the cutoff frequency of the filter, f_{-3dB}=1/(2πR_{cell}C_{cell}), will actually increase during electroporation. Given the typical small values of C_{cell}, measuring f_{-3dB} shift as a means of detecting electroporation may even may improve system sensitivity, particularly for cell layers with a low equivalent resistance. Estimation of R_{cell} or f_{-3dB} requires information at a number of distinct frequencies. Therefore, a sum of the sinusoidal pulses described above, where the frequency of the sinusoid used to generate each individual pulse is unique, can be used. The frequencies may be chosen such that an integer number of periods of each sinusoid is completed in the duration of pulse; for example, the frequencies may be separated by a factor of two. The amplitude of the resultant pulse is defined as the magnitude of the maximum excursion of the summation. For the sake of clarity, references to such summations of sinusoidal pulses will be henceforward referred to as simply sinusoidal pulses and figures referring to summations of sinusoidal pulses will depict a single frequency.

As described above for step pulses, contiguous sinusoidal pulses of varying amplitudes can be useful (FIG. 4.d). The low amplitude pulse (38) preceding the electroporation pulse (39 and 40) allows measurement of the impedance of non-electroporated cells. This measurement serves as comparison for the impedance measured during the electroporation pulse (39 and 40); a decrease in impedance during the electroporation pulse as (39 and 40) compared to that measured during the pre-electroporation pulse (38) indicates that electroporation has taken place. The first part of the electroporation pulse (39), is intended to initiate electroporation. The second part of the electroporation pulse (40) has a lower amplitude than the first part (39), and is intended to maintain electroporation. The low amplitude pulse following electroporation (41) allows assessment of the recovery of cells from electroporation; an increase in impedance during the post-electroporation pulse (41) as compared to that measured during the electroporation pulse (39 and 40) indicates that the cells have begun to recover from electroporation.

The step pulse technique can be combined with a sinusoidal component. This may be desirable in the case where the step pulses offer the most efficient electroporation for a given cell type, but the where the sinusoid, for the reasons described above, provides a superior impedance measurement. Such a pulse can be realized through the summation of a low amplitude (20-50 mV) sinusoid with the electroporation step pulses (39 and 40) shown in FIG. 4.c. The resultant pulse is shown in FIG. 4.e. Low amplitude sinusoidal pulses (42 and 45) are used for measurement before and after electroporation. Step pulses with a superimposed sinusoid (43 and 44) accomplish electroporation. The first part of the electroporation pulse (43), is intended to initiate electroporation. The second part of the electroporation pulse (44) has a lower amplitude than the first part (43), and is intended to maintain electroporation.

### Device for Controlled Electroporation in Tissue

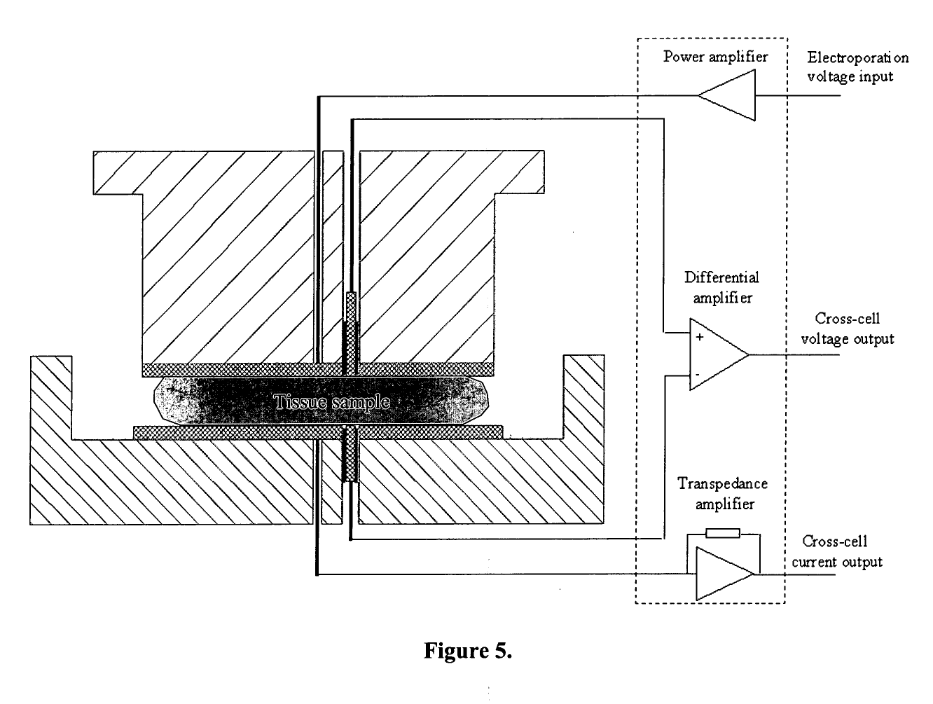

The device described above can also be adjusted to control electroporation in tissue, as shown in FIG. 5. In a typical electroporation procedure for tissue, the tissue sample is placed on the cup membrane (10), and the cup is placed between the two electroporation electrodes (3 and 15) as shown in FIG. 5, for in vitro electroporation. The tissue sample should be sized such that it covers the majority of the membrane (10). Alternately, or additionally, the tissue sample may be allowed to culture on the membrane (10) such that it attaches and spreads to cover the membrane (10) fully. An electrolyte is introduced to generate good contact between the tissue and the electrodes. Then, electrical pulses are applied to the tissue through the two electroporation electrodes (3 and 15) which are connected to the power amplifier (21) and the transimpedance amplifier (22). Measuring the electrical current through this electrical circuit is dependent on the overall and average degree of electroporation that the cells in the tissue sample between the electrodes experience. Once the cells are electroporated, there shall be increased electrical current flow through the cells and the magnitude of the electrical current becomes dependent on the degree of electroporation of the cells in tissue. This cross-cell electrical current can be measured with the transimpedance amplifier (22) and can be used to monitor the process of electroporation of the cell membranes. In addition to the current measurement, the two inserted probe electrodes (5 and 17) are used to precisely measure the voltage drop across the tissue during the electroporation process. The electrodes (5 and 17) are connected to the high input-impedance amplifier (23). Thus, since no current flows through these electrodes (5 and 17), there is no voltage drop over the electrode-electrolyte interfaces, and the differential voltage between them provides an accurate measurement of the voltage across the tissue. Precise electrical impedance of the tissue is thus calculated from cross-tissue voltage measurement with the probe electrodes (5 and 17) and cross-current measurement with the circuit attached to the electroporation electrodes (3 and 15). The impedance measurement reveals the degree of electroporation of the cells in tissue since cell membrane impedance is directly dependent on the extent of membrane electroporation. In addition to monitoring the electroporation, the electrical current measurement as well as membrane impedance measurement can be used as feedback for fine-tuning of electroporation pulses to achieve highly controlled electroporation of the cells in tissue.

### EXPERIMENTAL RESULTS

### Materials

Cells—Various types of cells were examined, including epithelial cells (such as MDCK cell line), fibroblast cells (such as NIH 3T3 cell line), lymphocytes (such as BCBL-1 cell line) and primary cells (such as skeletal satellite cells). Cell layers with desirable confluence were formed on various porous cell inserts from Millipore, Coming or BD Biosciences either by 1) growing cells on the porous inserts for various length of time (from a few hours to several days, depending on the cell type), or 2) by sucking cells in pores with pressure, as described previously.

Tissue—Tissue samples were obtained by slicing fresh mouse liver to a thickness ranging from 1 mm to 4 mm. Then a disk of liver was obtained by pressing a sharp circular tube onto the sample to trim the excess tissue. The resulting sample was then placed in the device for measurement. For negative controls we used livers that were kept prior to resection in a refrigerator at 4 C for three days.

### Electrical Parameters Study

Cells—Inserts with adherent layers of cells were placed into the configuration shown in FIG. 1a, medium was collected, cells were washed with PBS (phosphate buffered solution) and electroporation buffer (PBS or cell culture medium) was introduced to ensure good contact between the electrodes and both sides of the confluent cell layer. The electrical impedance of the sample was measured, after which a series of electroporation pulses were applied and the electrical data recorded.

Tissue—The tissue layer was placed between the electroporation electrodes of the device shown in FIG. 5. Electroporation buffer was added to ensure good contact between the electrodes and the tissue.

### Transfection Study

To assess the efficacy of the controlled electroporation and its ability to introduce various substances into cells, we have transfected cells with a variety of molecules, including fluorescent dyes (YOYO-1 and PI dyes), small and large DNA (such as GFP and MyoD genes), siRNA and antibodies, none of which are permeable to cell membranes under normal conditions. In our experiments, the reagent was mixed with electroporation buffer at desirable concentrations, and then introduced to the cell culture inserts where cell layer was formed. Delivery of those reagent molecules was enabled by electroporating the cell layer using the methods described above. Transfection expression was evaluated at various time points following electroporation, depending on how long it took for the expression to occur (immediate results are obtained using fluorescent dyes, one to two days are required for gene expression)

### Electroporation Electrical Measurement Results

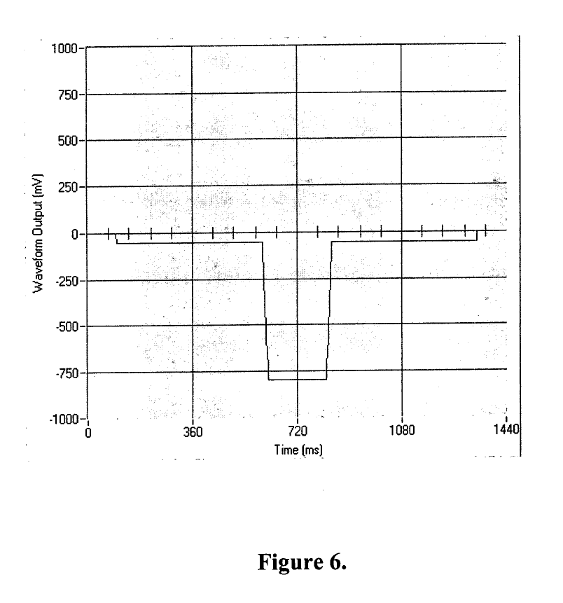

FIG. 6 shows a typical three-step electrical pulse, as depicted in FIG. 4b, used to study the process of electroporation in cell layers and tissue samples. It consists of three contiguous step pulses. The amplitude of the first step pulse is significantly below what is required to produce electroporation; it is used to probe the electrical impedance of the cells or tissue prior to electroporation. The second step pulse was varied in amplitude until a change in the electrical impedance of the cells was detected, indicating occurrence of electroporation. According to our invention, the occurrence of electroporation should result in a decrease in the electrical impedance of the cells, while electrical pulses which do not produce electroporation will not affect the electrical impedance of the cells. It should be noted that the polarity of the pulse was chosen such that the top electroporation electrode was at a lower potential than the bottom electrode, in order to facilitate the insertion of negatively charged molecules (such as DNA plasmids) into the cells through electrophoresis. The third electrical pulse has the same amplitude as the first. The impedance measured during the third pulse was used to determine if the electroporation was reversible or not. In our experiments we studied the effect of several sets of three contiguous step pulses, separated by various intervals of time.

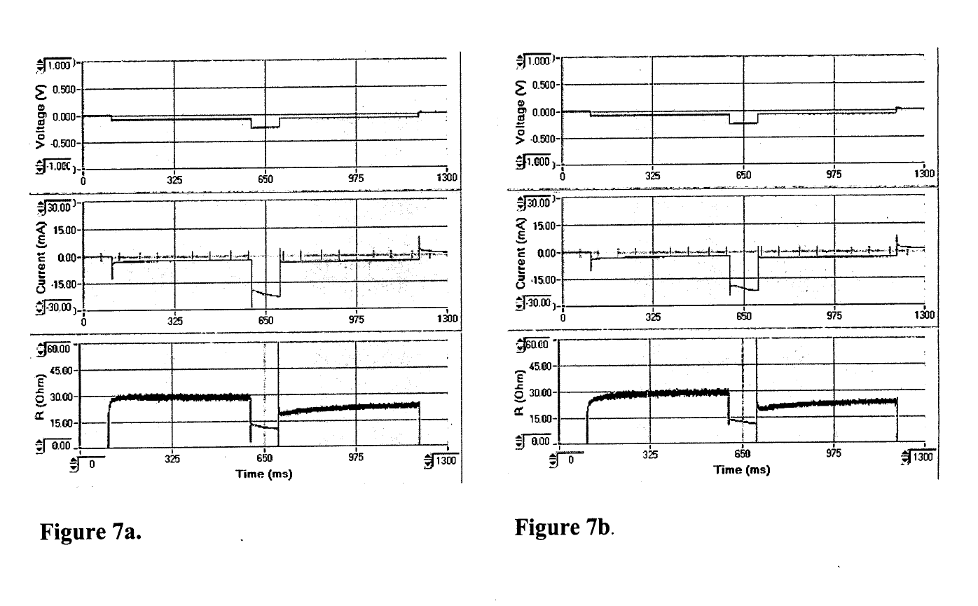

FIGS. 7.a, 7.b illustrate a sequence of electroporation pulses applied to satellite cells. The top graph in each figure shows the voltage across the cell layer in response to the three-step electroporation pulse described above; the first voltage step corresponds to the pre-electroporation impedance measurement pulse (50 mV/500 ms), followed by the electroporation step (300 mV/100 ms) and finally the post-electroporation impedance measurement pulse (50 mV/500 ms). The middle graph shows the current through the cell layer. Again, it should be noted that the current is negative and that the current during the middle electroporation pulse is larger than the current before and after the electroporation pulse. The bottom graph is the most important and illustrates the impedance of the cell layer. It should be noted that in all the figures, the cell layer impedance during the pre-electroporation measurement pulse is constant. In our experiments we have found that the impedance measured remains the same for pulses with increasing amplitude until a threshold is reached. However, when the amplitude of the pulse reached a threshold value, we would observe a significant drop in the electrical impedance, similar to the drop shown in FIGS. 7.a-b during the second, higher-amplitude electroporation step pulse. It is very interesting to note that the impedance decreases gradually throughout the electroporation portion of the pulse, which is consistent with the theory of electroporation. FIG. 7.b, which depicts an electroporation pulse applied at one minute after the first, indicates that the cell membrane essentially seals and returns to its original impedance within the one minute interval.

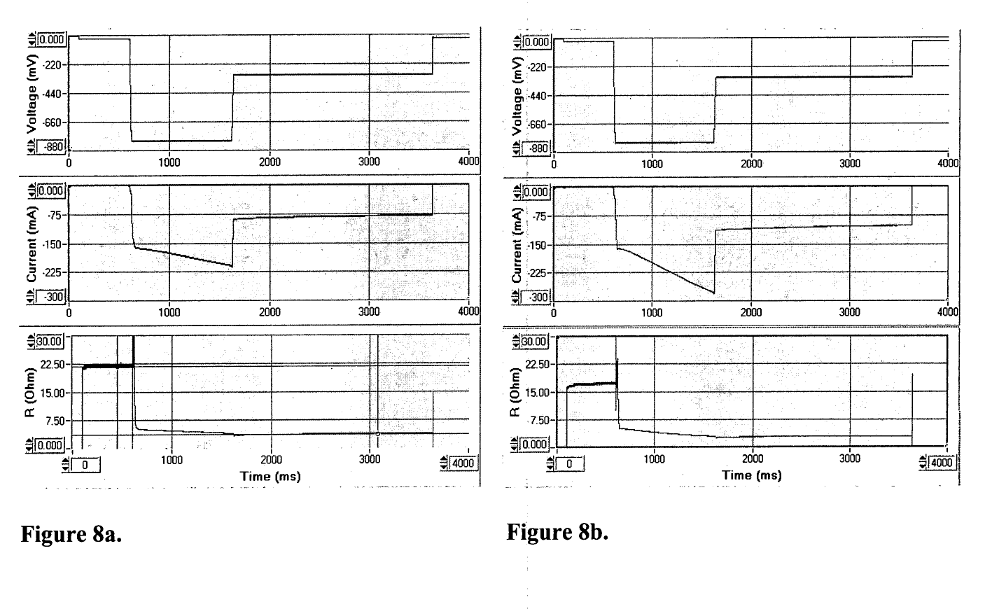

FIGS. 8 shows electroporation of cells using a 4-step electroporation pulse as depicted in FIG. 4c. It can be seen that the electroporation portion of the 4-step pulse consists of a 800 mV/1 sec main electroporation pulse, which was used to initiate electroporation, and a 300 mV/2 sec “maintaining” pulse, which was used to keep the high permeability state of the electroporated cells and to facilitate cross-membrane transfer of charged molecules via electrophoresis. As can be seen in the impedance plot, the impedance of the cell monolayer dropped significantly when the 800 mV pulse was imposed (from 22 ohms to 3.6 ohms) due to electroporation. When the pulse amplitude reduced to 300 mV (the maintaining pulse), the impedance of the cell layer still remain low at approximately 3.6 ohms, indicating the cells were kept at a highly permeable state by the low post-electroporation pulse. FIG. 8.b shows the data from an identical pulse applied a minute later. It clearly shows that the cell recovered during this one minute interval as the initial impedance obtained with the second pulse went back to about 17.7 ohms, which was significantly higher than the impedance of the cells in electroporated state. FIG. 8.b also illustrated the effect of the maintaining pulse, which kept the impedance of cells low after the cells were first electroporated by the 800 mV pulse.

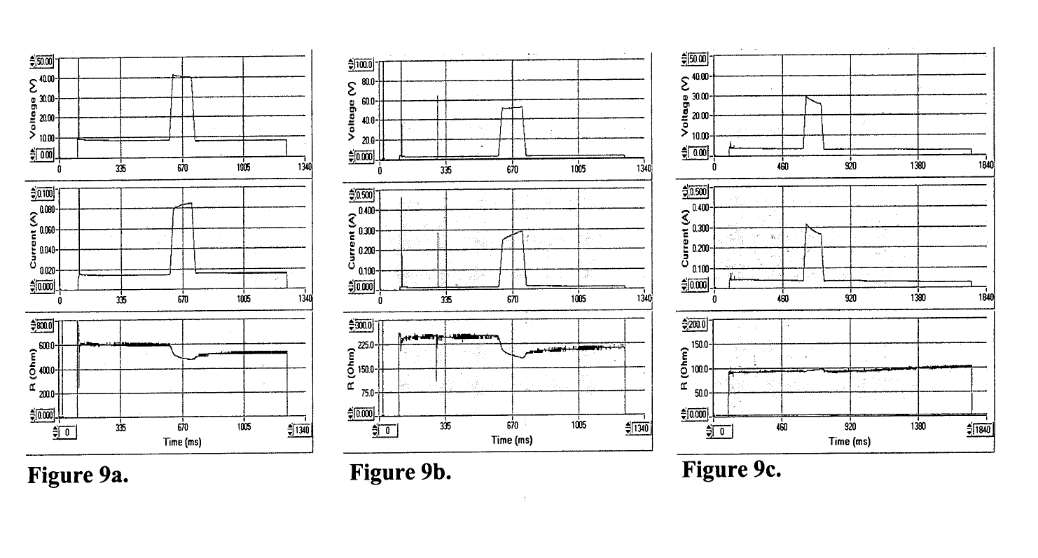

FIGS. 9a and 9b illustrate the typical behavior of fresh liver tissue during electroporation. It is evident that in response to the three-step pulse electroporation protocol, the tissue exhibits the same behavior as the layer of cells. Obviously the impedance of the layer of tissue is higher than that of the layer of cells. However, it also shows no change in impedance during the first portion of the pulse, which does not induce electroporation. Then, during the second pulse, which induces electroporation, the impedance drops. During the third pulse it returns to its initial value. FIG. 9.c shows the typical behavior of dead tissue. It can be seen that the impedance of the tissue slice is significantly lower than that of fresh tissue because dead cells have lower impedance than living cells as their membranes are impaired. It can also be clearly seen that the impedance of the dead tissue remained fairly constant during the entire pulse, indicating there was no further permeabilization in the impaired dead cell membranes even when high electrical pulses are applied. Thus, the change in impedance with electroporation is the hallmark of live cells and is what makes it possible to control the process of electroporation in live tissue, as claimed in this invention.

### Electroporation Efficiency and Cell Viability Assessment

Extensive experiments were performed to evaluate electroporation efficiency using the apparatus described above. Cell viability analysis was also carried out to assess the degree of damage to cells due to electroporation using our methods.

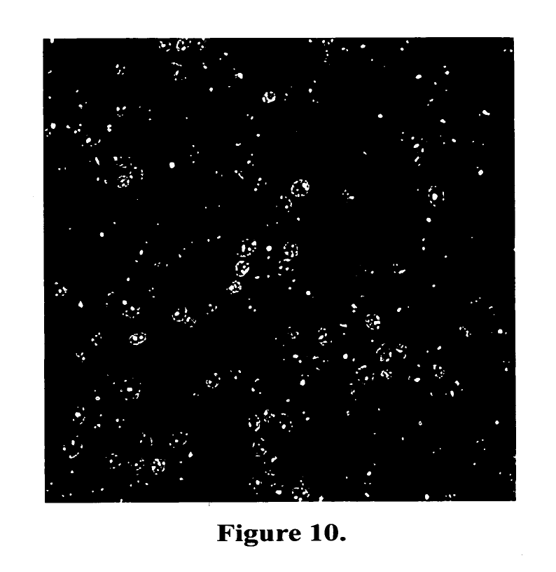

FIG. 10 shows illustrates the introduction of propidium iodide (PI), a fluorescent DNA stain that can not penetrate the membranes of normal cells, using our apparatus and method. Mandin Darby Canine Kidney (MDCK) cells were grown on a porous cell insert (Corning) for three days to form a confluent cell monolayer. 5 uL PI was added in PBS electroporation buffer, then three three-step pulses (FIG. 6) with 600 mv/300 ms electroporation pulses were applied at 1 minute interval to electroporate the cells in order to introduce the membrane impermeant PI into the cells. FIG. 10 was taken with a scanning fluorescent microscope under 20× objective. From the image, more than 90% cells in the monolayer were stained (red cells) indicating that more than 90% cells were effectively electroporated. In fact, we consistently achieved high electroporation efficiency (from 70% to nearly 100%) with this method on a variety of cells. The electroporation efficiency depends not only on electroporation pulses, but also on the confluence of the cell layer, which was explained in our previous sections.

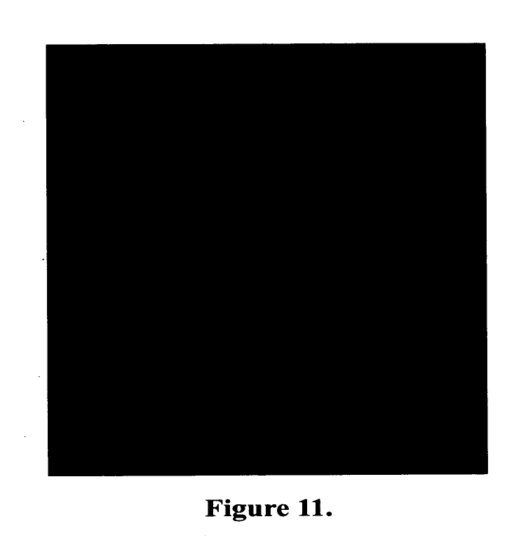

Cell viability after electroporation was assessed by adding membrane impermeant fluorescent dyes (such as PI, EthD-2 and YOYO-1) to cell buffer after electroporation pulses. The dyes are commonly used to mark dead cells because dead cells can not exclude the dye molecules due to their impaired membranes. FIG. 11 shows MDCK cells stained with PI after the typical procedures used to obtain electroporation. The nearly completely dark image indicated that there were virtually no dead cells (dead cells should appear in red color) after electroporation, meaning the electroporation didn't induce any noticeable membrane damages due to irreversible electroporation, which is commonly associated with traditional electroporation apparatuses. In addition to MDCK cells, we also performed such viability analysis on other cells, and we consistently achieved cell viability of more than 95% under our typical electroporation conditions.

### Gene Transfection

To evaluate the efficiency of gene transfection using our methods, we introduced two types of genes, GFP reporter gene and MyoD gene into various cell types. Typically, 5 ug DNA plasmids were mixed with electroporation buffer, and both three-step and four-step pulses (FIG. 4b and FIG. 4c) were applied to electroporate cells for gene transfer. The polarity of applied pulses was set to be negative in order to facilitate insertion of negatively charged DNA into cells through electrophoresis. Expression of the genes was typically evaluated at 24-72 hours after electroporation. GFP expression was observed under green filter fluorescence microscopy. MDCK cells were viewed on the same porous membrane on which they were cultured. Treated fibroblasts and satellite cells were trypsinized and centrifuged at 1800 rpm for 10 minutes at RT. Pellet was suspended in cold PBS with glucose (2.5 gr/L), and cytospinned at 500 rpm for 15 minutes on glass microscope slides.

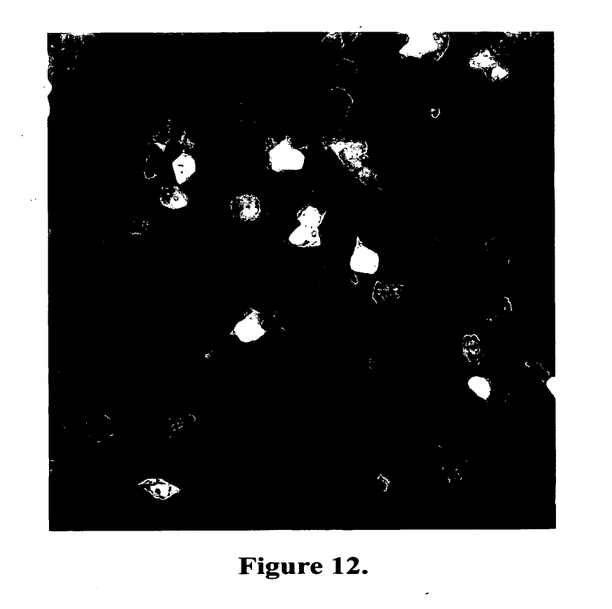

FIG. 12 shows transfection of GFP reporter gene in a differentiated MDCK monolayer. From the image, more than 95% MDCK cells expressed the reporter gene (cells in green fluorescence), comparing to at most 16% transfection rate reported using other methods, such as lipotransfection.

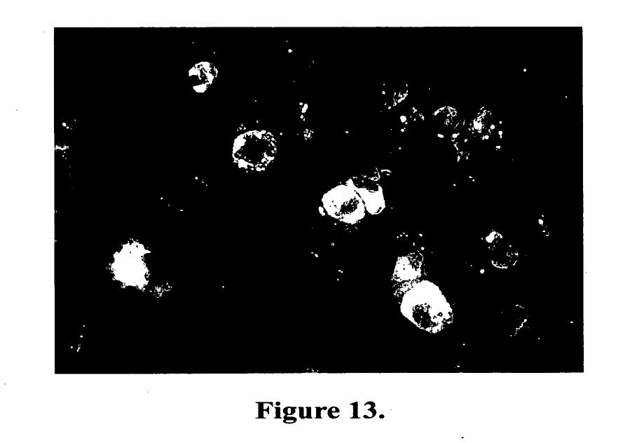

FIG. 13 shows transfection of GFP gene in primary satellite stem cells. More than 95% of cells were positively transfected. We also found that in every experiment in which the impedance measurements indicated electroporation we had expression of the gene, and no expression (0%) in the negative controls where there was no electroporation.

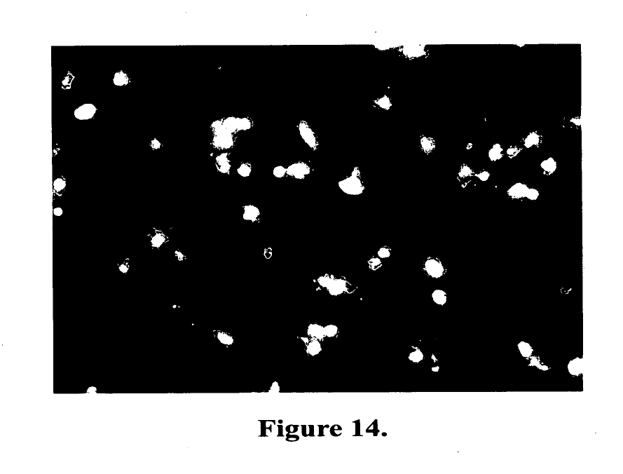

FIG. 14 shows transfection of GFP gene in mouse skin fibroblast cells (NIH 3T3 cell line), which indicates a transfection efficiency of more than 90%.

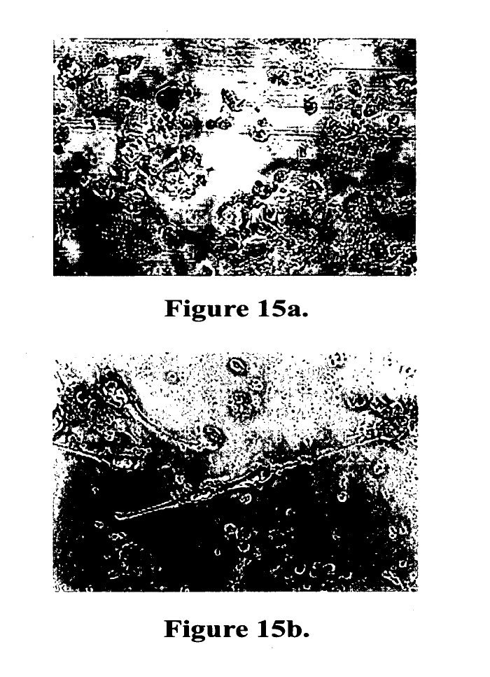

FIG. 15 shows the transfection of large MyoD genes (˜40 Kb), which converts fibroblast cells into myotube muscle cells, using our apparatus. Through serum deprivation, the MyoD treated fibroblasts differentiated and fused into multinucleated nascent myotubes that were stained positive for sarcomeric actin/myosin. These morphologic and myogenic changes were observed in all impedance-monitored electroporation and absent in control fibroblasts. FIGS. 15a and 15b illustrate the normal fibroblast cells and the converted myotubes that were induced by transfection through electroporation of fibroblasts.

### Transfection of siRNA

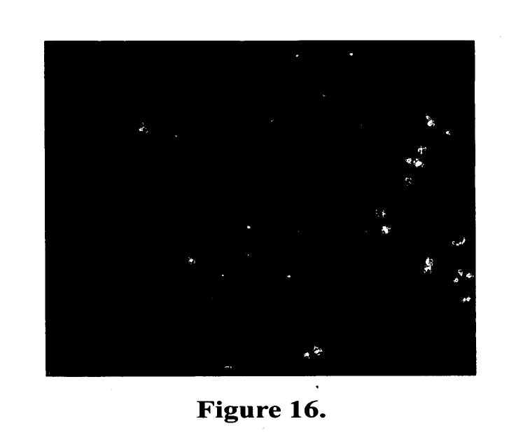

FIG. 16 demonstrates our apparatus's capability of delivering siRNA (small interfering RNA) into cells. In the experiment, fluorescenated siRNA (siRNA-FITC from Qiagen, San Diego) was added in electroporation buffer, and then MDCK cells were electroporated using the method and conditions previously described. After electroporation, MDCK cells were detached from cell inserts by trypsinization, re-suspended and loaded onto a glass slide for fluorescence microscopy. Cells that were uploaded with fluorescenated siRNA molecules appeared in green under fluorescent microscope. By visual inspection, we estimated the efficiency of siRNA introduction was consistently more than 90%, as shown in the figure.

### Transfection of Antibodies

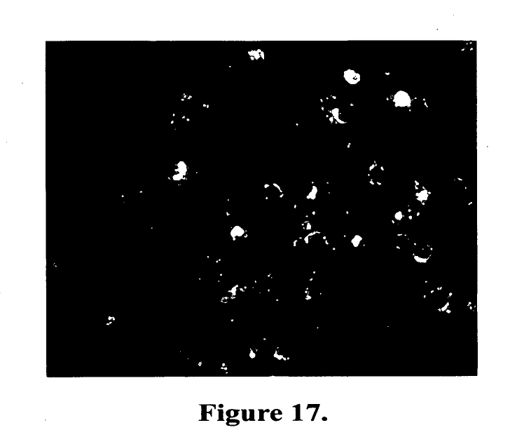

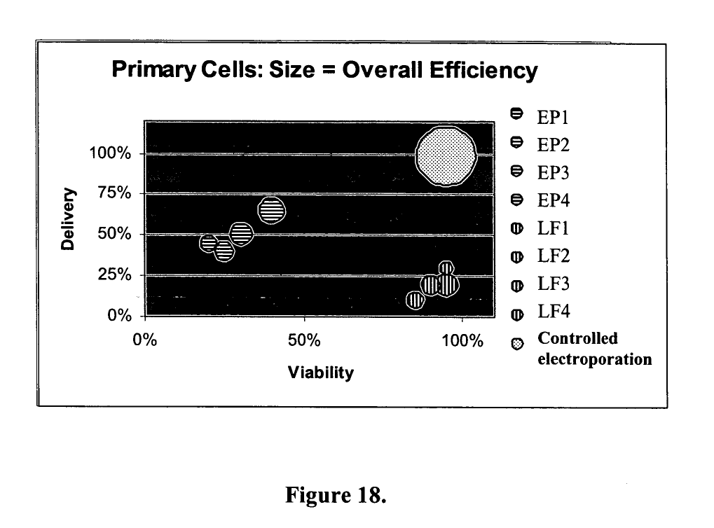

To demonstrate our apparatus's capability of transfecting cells with antibodies, we performed experiments to introduce a fluorescenated antibody (BCL-FITC) into MDCK cells. Experiment protocol was similar with the one for siRNA transfection experiment. FIG. 17 shows the fluorescent image of the transfected cells. Cells that were successfully delivered with BCL-FITC antibodies appeared in green fluorescence in the image. The image showed that the efficiency of antibody transfection reached nearly 100% with our apparatus.

### DEFINITIONS

The phrase “characterize cell” is intended to include the assessments including membrane integrity; the effectiveness with which a cell blocks a pore; cell health; and cell viability, and any combination thereof.

The phrase “characterize electroporation” is intended to include determinations of the onset, the extent and the duration of electroporation, as well as an assessment of the recovery of cell membranes after electroporation, and any combination thereof.

The term “charged entity” shall include any positively or negatively charged molecule or polymer, and can be of biological origin, such as a peptide, a protein or a nucleic acid, and any combination thereof.

The term “biological entity” refers to any entity with a bilipid membrane, and includes biological cells, artificial cells or lipid vesicles and any combination thereof. Without loss of generality, the term “cell” shall refer to such a biological entity. Again, without loss of generality, the term “cell layer” will include cases in which cells cover the membrane fairly uniformly, in one or more layers, or when they preferentially congregate over micro pores. Other examples of cell layers include biological tissue, biological tissue slices, spheroids, cultures of non-contact-inhibited adherent cells, adherent cell monolayers, collections of cells and spheroids deposited by some mechanical means, and cells and spheroids preferentially blocking micro pores, and any combination thereof.

The term “impedance” is used herein to mean a ratio of current to voltage. The term “resistance” is also used to mean a ratio of current to voltage.

1. A method, comprising the steps of:
creating an electrical charge differential between a first point and a second point separated from the first point by an electrically conductive medium comprising a biological cell;
substantially blocking electrical current from between the first point and the second point except through the biological cell; and
imposing a substantially uniform electric charge differential across the biological cell.

2. The method of claim 1, wherein the electrical charge differential induces electroporation of the biological cell membrane.

3. The method of claim 1, wherein the imposed electrical charge differential induces mass transport across the cell membrane via diffusion.

4. The method of claim 1, wherein the imposed electrical charge differential induces mass transport of a charged entity across the cell membrane via electrophoretic force, electrokinetic or electroosmotic flow.

5. The method of claim 1, wherein the electrically conductive medium comprises a plurality of cells.

6. The method of claim 5, wherein the biological cell comprises primary cells.

7. The method of claim 6, wherein the cells are selected from the group consisting of nerve cells and stem cells.

8. The method of claim 1, further comprising: measuring a first electrical parameter between the first and second points; and adjusting a second electrical parameter based on the measuring of the first electrical parameter.

9. The method of claim 8, wherein the electrical charge differential induces electroporation of the cell membrane.

10. The method of claim 8, wherein the imposed electrical charge differential induces mass transport across the cell membrane via diffusion.

11. The method of claim 8, wherein the imposed electrical charge differential induces mass transport of a charged entity across the cell membrane via electrophoretic force, electrokinetic or electroosmotic flow.

12. The method of claim 8, wherein the electrically conductive medium comprises a plurality of cells.

13. The method of claim 12, wherein the cell comprises primary cells.

14. The method of claim 13, wherein the cells are selected from the group consisting of nerve cells and stem cells.

15. The method of claim 8, wherein the first electrical parameter is selected from the group consisting of current, voltage and electrical impedance and the second electrical parameter is selected from the group consisting of current, voltage and a combination of current and voltage.

16. The method of claim 15 using impedance to characterize the cell.

17. The method of claim 15, using impedance to characterize electroporation.

18. A method, comprising the steps of:
sending an electrical current between a first point and a second point separated from the first point by an electrically conductive medium comprising a biological cell suspended therein;
substantially blocking electrical current from between the first point and the second point except through the biological cell; and
imposing a substantially uniform electric field across the biological cell.

19. The method of claim 18, wherein the applied electrical potential is between about 0 and about 24 volts.

20. The method of claim 18, further comprising: measuring a first electrical parameter in the medium; and adjusting a second electrical parameter based on the measuring of the first electrical parameter.

21. The method of claim 20, wherein the first electrical parameter is selected from the group consisting of current, voltage and electrical impedance and the second electrical parameter is selected from the group consisting of current, voltage and a combination of current and voltage.

22. The method of claim 21, using impedance to characterize the amount of electroporation induced by the applied electric potential.

23. The method of claim 22, using stepwise increments of applied electric potential to determine the threshold of electroporation.

24. The method of claim 23, using an algorithm to determine the applied electric potential required for electroporation.

25. The method of claim 24, determining the applied electric potential required for a minimum effective threshold of electroporation for a plurality of cells.

26. Apparatus for the manipulation of a biological cell, the apparatus comprising:
an electric cell containing an internal support capable of holding a biological cell and an internal barrier of a material substantially impermeable to electric current, the barrier positioned to restrict electric current flow in the electric cell to a flowpath crossing the internal support and through any biological cell held thereby; and
means for imposing a voltage across the electric cell and for monitoring the value of current, voltage or electrical impedance and using the value to regulate the current, voltage or a combination of current and voltage; and
means for imposing a substantially uniform electric field across the biological cell held thereby.

27. The apparatus of claim 26 in which the electric field across the biological cell is preferably between 0 and 24 volts.

28. The apparatus of claim 26 in which the electrode material exposed to the biological cell medium is composed of silver/silver chloride.

29. The apparatus of claim 26 is which the electrodes are of sufficient size and proximity to the barrier to create a substantially uniform electric field across the barrier.

30. The apparatus of claim 26 further comprising means for immobilizing the biological cell between two internal supports.

31. The apparatus of claim 26 in which the barrier divides the interior of the electric cell into first and second electrode chambers and the internal support is an opening in the barrier smaller in width than a biological cell.

32. The apparatus of claim 31 further comprising means for immobilizing the biological cell through adhesion or affinity immobilization to form an effective resistive seal over the opening.

33. The apparatus of claim 31 further comprising means for immobilizing the biological cell through pressure differential to form an effective resistive seal over the opening.

34. The apparatus of claim 31, wherein the openings have diameters greater than about 0.1 um and less than about 10 um.

35. The apparatus of claim 31, wherein the opening densities are greater than about 1×10³/cm² and less than about 1×10¹⁰/cm².

36. The apparatus of claim 31 in which the barrier and internal support are combined in a semi-porous membrane.

37. A method for electroporation of a biological cell, comprising the steps of:
sending an electrical current between first and second electrodes separated by an electrically conductive medium comprising a biological cell suspended therein;
substantially blocking electrical current from between the first and second electrodes except through the biological cell through the use of a semi-porous membrane; and
measuring a first electrical parameter in the medium; and adjusting a second electrical parameter based on the measuring of the first electrical parameter; and
imposing a substantially uniform electric field across the biological cell.

38. The method of claim 37, wherein the electrically conductive medium comprises a plurality of cells.

39. The method of claim 38, wherein the imposed electric field achieves a specified efficiency of electroporation while maintaining greater than 90% cell viability.

40. The method of claim 39, wherein the electroporation efficiency is greater than about 90% and less than about 100%.

41. The method of claim 39, wherein the electroporation efficiency is greater than about 80% and less than about 100%.

42. The method of claim 39, wherein the electroporation efficiency is greater than about 70% and less than about 100%.

43. The method of claim 39, wherein the electroporation efficiency is greater than about 60% and less than about 100%.

44. The method of claim 37, wherein a pore blocking method is employed to effectively cover more than about 90% but less than about 100% of the pores of the membrane for a sub-confluent layer of biological cells.

45. The method of claim 37, wherein a pore blocking method is employed to effectively cover more than about 80% but less than about 100% of the pores of the membrane for a sub-confluent layer of biological cells.

46. The method of claim 37, wherein a pore blocking method is employed to effectively cover more than about 70% but less than about 100% of the pores of the membrane for a sub-confluent layer of biological cells.

47. The method of claim 37, wherein a pore blocking method is employed to effectively cover more than about 60% but less than about 100% of the pores of the membrane for a sub-confluent layer of biological cells.

48. The method of claim 37, wherein a pore blocking method is employed to effectively cover more than about 50% but less than about 100% of the pores of the membrane for a sub-confluent layer of biological cells.

49. The method of claim 37, wherein a pore blocking method is employed to effectively cover more than about 40% but less than about 100% of the pores of the membrane for a sub-confluent layer of biological cells.

50. The method of claim 37, wherein a pore blocking method is employed to effectively cover more than about 30% but less than about 100% of the pores of the membrane for a sub-confluent layer of biological cells.

51. The method of claim 37, wherein a pore blocking method is employed to effectively cover more than about 20% but less than about 100% of the pores of the membrane for a sub-confluent layer of biological cells.
